(19) United States
(12) Patent Application Publication
(10) Pub. No.: US 2010/0179303 A1
(43) Pub. Date: Jul. 15, 2010

(54) SITE-SPECIFIC CONJUGATION OF LIGANDS TO NANOPARTICLES

(75) Inventors: Arutselvan Natarajan, Sacramento, CA (US); Sally J. Denardo, El Macero, CA (US); Gerald L. Denardo, El Macero, CA (US); Cordula Gruettner, Rostock-Warnemuende (DE)

Correspondence Address: SHIMOKAJI & ASSOCIATES, P.C., 8911 RESEARCH DRIVE, IRVINE, CA, 92618, US

(73) Assignee: THE REGENTS OF THE UNIVERSITY OF CALIFORNIA, OAKLAND, CA (US)

(21) Appl. No.: 12/597,866

(22) PCT Filed: Apr. 25, 2008
(86) PCT No.: PCT/US08/61676

§ 371 Date: Mar. 1, 2010

Related U.S. Application Data
(60) Provisional application No. 60/914,613, filed on Apr. 27, 2007.

(51) Int. Cl. C07K 14/00; C07K 2/00; C07K 16/00

(52) U.S. Cl. 530/300; 530/402; 530/391.1; 530/391.5; 977/773

(57) ABSTRACT

A new radioconjugate NP has been developed using recombinant antibody fragments, di-scFv-c (111 In-DOTA-di-scFv-NP) for imaging and therapy of anti MUC-1 10 expressing cancers since aberrant MUC-1 Is abundantly expressed on the majority of human epithelial cancers. Selection and engineering of anti-MUC-1 di-scFv-c (50 kDa) was generated to link NP-M (maleimide). DOTA chelate was conjugated with di-scFv-c for the radiometal chelation to trace the RINP at in vivo. This RINP preparation can provide uniquely high tumor cell binding NP for AMF driven tumor hyperthermia.

### CROSS-REFERENCE TO RELATED APPLICATION

This application claims benefit under 35 U.S.C. §119(e) of U.S. Provisional Application having application No. 60/914,613 filed Apr. 27, 2007, which is hereby incorporated by reference herein in its entirety.

### STATEMENT REGARDING FEDERALLY SPONSORED RESEARCH OR DEVELOPMENT

This invention was made with Government support under NIH grant CA47829 awarded by NCI. The Government has certain rights in this invention.

### BACKGROUND OF THE INVENTION

Increasing the temperature in living tissues to 42-46° C. leads to cell death by inactivation of normal cellular processes; triggering cell death by temperatures above 46° C. has been shown to be associated with extensive necrosis known as thermal ablation. (Streffer C and D van Beuningen. The biological basis for tumor therapy by hyperthermia and radiation. Recent Results Cancer Res 1987; 104:24. Overgaard J. Effect of hyperthermia on malignant cells in vivo. A review and a hypothesis. Cancer 1977; 39:2637. DeNardo S J, G L DeNardo, L A Miers, et al. Development of tumor targeting bioprobes ((111)In-chimeric L6 monoclonal antibody nanoparticles) for alternating magnetic field cancer therapy. Clin Cancer Res 2005; 11:7087s.)

Inducing heat in cancer cells through external application of alternating magnetic field (AMF) for activation of targeted nanoparticles (NPs) has been demonstrated to be a possible method for treating cancer. The degree of micro-heating induction by an external AMF application depends on several factors including the concentration of nanoparticles in the cancer and the ferromagnetic properties of the NPs. Functionalized iron oxide nanoparticles have been reported that demonstrated heat induction by AMF (Tanaka K, A Ito, T Kobayashi, et al. Heat immunotherapy using magnetic nanoparticles and dendritic cells for T-lymphoma. J Biosci Bioeng 2005; 100:112. DeNardo S J, G L DeNardo, A Natarajan, et al. Thermal dosimetry predictive of efficacy of 111In-ChL6 nanoparticle AMF-Induced thermoablative therapy for human breast cancer in mice. J Nucl Med 2007; 48:437. Ito A, K Tanaka, H Honda, et al. Complete regression of mouse mammary carcinoma with a size greater than 15 mm by frequent repeated hyperthermia using magnetite nanoparticles. J Biosci Bioeng 2003; 96:364.)

Monoclonal antibodies (MAb) and fragments are known tumor-targeting agents and can be linked to NPs to direct them to cancer cells. Radiochelators can also be tagged to the NP or MAb to track their distribution and quantification. In a previous paper it has been demonstrated that ¹¹¹In-DOTA-ChL6-NP targeted human breast cancer xenografts in vivo. (DeNardo S J, G L DeNardo, L A Miers, et al. Development of tumor targeting bioprobes ((111)In-chimeric L6 monoclonal antibody nanoparticles) for alternating magnetic field cancer therapy. Clin Cancer Res 2005; 11:7087s.) Heat was induced by external AMF applications, achieving measurable therapeutic response with tolerable toxicity. (DeNardo S J, G L DeNardo, A Natarajan, et al. Thermal dosimetry predictive of efficacy of 111In-ChL6 nanoparticle AMF-Induced thermoablative therapy for human breast cancer in mice. J Nucl Med 2007; 48:437.)

Multivalent forms of scFv, di-scFv, dimeric scFv and tri-scFv molecules have been studied for imaging and cancer therapy. (A Natarajan, C Y Xiong, H Albrecht, et al. Characterization of Site-Specific ScFv PEGylation for Tumor-Targeting Pharmaceuticals. Bioconjug Chem 2005; 16:113. Wu A M and P D Senter. Arming antibodies: prospects and challenges for immunoconjugates. Nat Biotechnol 2005; 23:1137.) Recombinantly made di-scFv-c have exhibited a significant improvement in tumor targeting over the monovalent scFv. (Albrecht H and S J DeNardo. Update: recombinant antibodies: from the laboratory to the clinic. Cancer Biotherapy & Radiopharmaceuticals 2006; 21:285. Wu A M, W Chen, L Raubitschek, et al. Tumor Localization of anti-CEA single-chain Fvs: improved targeting by non-covalent dimers. Immunotechnology 1996; 2:21. Goel A, G W Beresford, D Colcher, et al. Divalent forms of CC49 single-chain antibody constructs in Pichia pastoris: expression, purification, and characterization. J Biochem (Tokyo) 2000; 127:829.)

Mucin-1 is a transmembrane molecule, expressed by most glandular epithelial cells. (Xiong C Y, Natarajan A, Shi X B, et al. Development of tumor targeting anti-MUC-1 multimer: effects of di-scFv unpaired cysteine location on PEGylation and tumor binding. Protein Eng Des Sel 2006; 19:359. Winthrop M D, S J DeNardo, H Albrecht, et al. Selection and characterization of Anti-MUC-1 scFvs intended for targeted therapy. Clin Cancer Res 2003; 9:3845s.) Several important features make MUC-1 an attractive molecule for targeting cancer. (Barratt-Boyes S M. Making the most of mucin: a novel target for tumor immunotherapy. Cancer Immunol Immunother 1996; 43:142. Moore A, Z Medarova, A Potthast, et al. In vivo targeting of underglycosylated MUC-1 tumor antigen using a multimodal imaging probe. Cancer Res 2004; 64:1821.)

As can be seen, there is a need for a delivery system and method for attaching a ligand, such as a polypeptide, to a NP without interfering with the binding site of the ligand. Such a ligand may be, for example an anti-MUC-1 targeting NP, containing a radioactive tracer that effectively binds MUC-1 expressing human cancer cells both in vitro and in vivo.

### SUMMARY OF THE INVENTION

According to one aspect of the present invention, a conjugate comprises a nanoparticle; and at least one ligand having a free thiol group, said free thiol group linked to said nanoparticle.

According to another aspect of the present invention, a conjugate comprises a ligand having dual specificity toward a tumor cell and toward a metal chelating group; and a nanoparticle attached to a free cysteine of said ligand.

According to a further aspect of the present invention, a conjugate comprises a nanoparticle; and at least five ligands conjugated to said nanoparticle through a thiol group of the ligand, each of the ligands comprising a bivalent molecule.

According to still another aspect of the present invention, a radio-immuno nanoparticle (RINP) comprises a nanoparticle; and a plurality of radiolabeled ligands attached to said nanoparticle through a thiol group of each of the ligands. such that said RINP has at least ten MUC-1 binding units.

These and other features, aspects and advantages of the present invention will become better understood with reference to the following drawings, description and claims.

### BRIEF DESCRIPTION OF THE DRAWINGS

FIG. 1 is a schematic of an RINP according to one embodiment of the present invention;

FIG. 2 is a table of the properties of RINP (¹¹¹In-DOTA-di-scFv-SPIO-NP) according to one embodiment of the present invention;

FIG. 3 is (¹¹¹In-DOTA-Bz-di-scFv/di-scFv-20 nm-NP) on 4-12% SDS PAGE spectrum, left: CB stained and right: scanned on a Fuji Imager according to one embodiment of the present invention;

FIG. 4 is (¹¹¹In-DOTA-Bz-di-scFv/di-scFv-20 nm-NP) on CAE 11 min and 45 min according to one embodiment of the present invention;

FIG. 5 is a plot of percent binding versus incubation time di-scFv-20 nm beads binding effect on HBT and DU145 according to one embodiment of the present invention;

FIG. 6 is a box plot of binding percent on cells versus binding effect on different time intervals according to one embodiment of the present invention;

FIG. 7a is a microscopic view of bioprobes binding with HBT cells according to one embodiment of the present invention;

FIG. 7b is a microscopic view of bioprobes binding with DU145 cells according to one embodiment of the present invention;

FIG. 8 is a Whole Body Autoradiograph (WBAR) according to one embodiment of the present invention;

FIG. 9 is a graph of blood and body clearance of RINP according to one embodiment of the present invention;

FIG. 10 is a box plot of RINP uptake by various organs according to one embodiment of the present invention;

FIG. 11 is a table of immunoreactivity of the RINP and NP using MUC-1 (+) and (−) cell lines according to one embodiment of the present invention;

FIG. 12 is a table particle characteristics/specification according to one embodiment of the present invention;

FIG. 13 is a table of conjugation of ¹¹¹In-DOTA-Bz-di-scFv with Nanomag®-D-Spio maleimide according to one embodiment of the present invention;

FIG. 14 is a flow chart of a method according to one embodiment of the present invention;

FIG. 15 is a table of QA and specific activity of the radio labeled Spio beads according to one embodiment of the present invention;

FIG. 16 is ¹¹¹In-DOTA-Bz-di-scFv/di-scFv-20 nm-Nanomag®-D-Spio, UCD121305-O2AN: CAE 11 and 45 min according to one embodiment of the present invention;

FIG. 17 is ¹¹¹In-DOTA-Bz-di-scFv/di-scFv-20 nm-Nanomag®-D-Spio beads on 4-12% PAGE spectrum, left: CB stained and right: scanned on a Fuji Imager according to one embodiment of the present invention;

FIG. 18 is 4-12% SDS PAGE spectrum scanned on a Fuji Imager according to one embodiment of the present invention;

FIG. 19a is a microscopic view of ¹¹¹In-DOTA-di-scFv/di-scFv-20 nm spiobeads with DU145 cells according to one embodiment of the present invention; and

FIG. 19b is a microscopic view of ¹¹¹In-DOTA-di-scFv/di-scFv-20 nm spiobeads with Raji cells according to one embodiment of the present invention.

### DETAILED DESCRIPTION OF THE INVENTION

The following detailed description is not to be taken in a limiting sense, but is made merely for the purpose of illustrating the general principles of the invention, since the scope of the invention is best defined by the appended claims.

As used herein, the term “nanoparticle” refers to a particle having a diameter of less than 500 nm and typically less than 100 nm. Examples of nanoparticles may include iron oxide particles, gold particles, liposomes, polyethylene glycol (PEG) and the like.

As used herein, the term “ligand” refers to any matter that may be attached to a nanoparticle. For example, a ligand may include polypeptides, proteins, antibodies, antibody fragments, single chain variable fragments and the like.

As used herein, the term “conjugate” refers to a complex formed between at least one nanoparticle and at least one ligand.

As used herein, the term “binding region” refers to the region of a ligand that may bind to a target. For example, in cancer treatment, the target may be MUC-1 expressing human cancer cells.

Broadly, an embodiment of present invention provides delivery systems and methods for attaching a ligand to a NP. The resulting conjugate may be, for example, a RadioImmunoNanoparticle (RINP) for cancer therapy. As a non-limiting example, the ligands of the present invention may comprise engineered antibody fragments, such as single chain variable fragments (scFv-cys) and bivalent molecules (di-scFv-cys), site-specifically conjugated to nanoparticles (e.g. maleimide activated 20 nm spiobeads). The recombinant antibodies fragments may have dual specificity toward a tumor-associated antigen (e.g. MUC-1) and toward a functional moiety such as a metal chelating group for radionuclide binding (DOTA).

The ligands of the present invention may be attached to the NP through a thiol present in the ligand. The thiol may be a naturally present in the ligand or may be engineered into the ligand. For example, the thiol may be part of the amino acid cysteine. The cysteine may be engineered into the ligand through various methods known in the art, such as recombinant engineering, chemical synthesis, and the like. In one embodiment of the present invention, the thiol does not interfere with the binding region of the ligand. The ligands of the present invention may be single valent, having one binding site, or there may be multiple binding sites on a single ligand.

The nanoparticles of the present invention may be any typical nanoparticle known in the art. The nanoparticles of the present invention may be coated as is known in the art. As a non-limiting example, the nanoparticles may be PEG coated dextran-iron oxide nanoparticles, as described in greater detailed. The nanoparticles may have any number of binding sites onto which to attach the ligands. Typically, the nanoparticles may be capable of binding from 1 to about 50 ligands thereto.

The conjugates of the present invention may have a variety of uses, especially a variety of medical uses for treating mammals, as is known in the art for typical (non-thiol bound, for example) antibody-NP conjugate systems. For example, the conjugates of the present invention may be useful for cancer targeting, biologic cross-linking, pretargeting cells for subsequent treatment, and the like.

Unlike the prior art in which monoclonal antibodies are conjugated to the nanoparticles, the present invention can include any ligand conjugated to the nanoparticles via a free thiol group. This allows more antigen binding units to be conjugated with minimal increase in bioprobe size.

Unlike the prior art that does not provide site-specific conjugation, the antibody fragments of the present invention may be genetically engineered to include a free sulfhydryl group (SH) for site-specific conjugation. For example, in one embodiment, the present invention can include a free cysteine (di-scFV-cys) for site-specific conjugation. The location of the engineered cysteine within the fragment may vary. For example, the free cysteine may be located after the fifth amino acid in the linker or near the carboxyl terminus of the fragment. The free cysteine can provide a specific site for thiol conjugation. The location of the cysteine may affect tumor binding and polyethylene glycol-maleimide (PEG-Mal) conjugation (PEGylation). This is unlike the prior art where random PEGylation of the fragments via amine groups can led to variations of structural conformation and binding affinity.

Embodiments of the present invention provide site-specific conjugation of di-scFv-cys (with and without radiometal attached) to maleimide activated nanoparticles (20 nm particles) allowing small binding units to minimize increase in nanoparticle size and each binding unit (scFv-c) to retain complete binding of its antigen binding site. This allows the integration of multiple binding units per nanoparticle with little increase in total particle size, increasing tumor binding by an order of magnitude (avidity) over nanoparticle antibody conjugates conjugated with the same total protein and size, and thus less binding sites and less retention of those attached. Multi functional scFv units can be used to provide nanoparticles capable of binding several antigen targets. This provides pretargeting capabilities for several isotope biological and drug therapies. This invention may be useful for in vivo alternating magnetic field (AMF) thermal ablation, hyperthermia therapy targeting, chemotherapy targeting, pretargeting radionuclide therapy, as well as NMR, SPECT and PET tumor imaging. Site specific conjugation allows preparation of high avidity homogenous nanoparticle conjugates, much superior to nanoparticle conjugates of antibodies and nanoparticles with non-site specific conjugated antibody fragments.

To develop second generation radioimmuno nanoparticles (RINPs), the targeting MAb has been replaced with anti MUC-1 di-scFv-cys; these di-scFv-cys provide more binding units per particle with little increase of final RINP size so as to enhance effective targeting. Maleimide nanoparticles (NP-M) and di-scFv-c were linked by a site-specific attachment to a free cysteine of the di-scFv engineered to minimize interference with the tumor binding site. Xiong C Y, Natarajan A, Shi X B, et al. Development of tumor targeting anti-MUC-1 multimer: effects of di-scFv unpaired cysteine location on PEGylation and tumor binding. Protein Eng Des Sel 2006; 19:359.)

### Materials and Methods

Carrier-free ¹¹¹In (MDS Nordion, Ontario, Canada) was purchased as indium chloride in 0.05 M HCl. A high-gradient magnetic field column separator (HGMF), and maleimide functionalized, PEG coated 20 nm dextran-iron oxide nanoparticles (Nanomag®-D-spio, 79-96-201) and COON functionalized PEG coated 20 nm dextran-iron oxide nanoparticles (Nanomag®-D-spio, 79-56-201) were obtained from micromod Partikeltechnologie GmbH (Rostock, Germany). 2-(4-morpholino) ethanesulphonic acid (MES), phosphate buffer solution (PBS), 1-ethyl-3-(3-dimethylaminopropyl)-carbodiimide HCl, N-hydroxysuccinimide, glycine (Sigma Chemical Co., St. Louis, Mo.), and 3,400 MW cutoff dialysis modules (Pierce, Rockford, Ill.) were purchased. DOTA-benzyl-NCS and DOTA-benzyl-NH₂ were purchased from Macrocyclics, Dallas, Tex., USA. Sephadex G25 and G75 were obtained from Pharmacia, Uppsala, Sweden. Radiolabeled DOTA-di-scFv was purified and analyzed using a Beckman Coulter System Gold 128 HPLC system with a radioactive detector (Raytest USA, Wilmington, N.C.). Quality assurance (QA) and protein concentration of the RIC used in conjugation with NP and RIC from HGMF column washings were determined using SEC 3000 columns with PBS buffer, of pH 7.0, eluted at 1 ml/minute. Radioactivity of cellulose acetate electrophoresis (CAE) (Gelman Sciences, Inc., Ann Arbor, Mich.) was performed using 0.05 M sodium barbital buffer, pH 8.6, and a current of 5 mA per strip was applied. Radioactivity profile was determined by scanning on a BAS-1800 phosphor imager exposed on imaging plate (IP) (Fuji Photo Film Co, Tokyo, Japan). The radioactivity resolution from IP was digitally converted to graph using Multi Gauge 2.1 software (Fuji Photo Film Co, Tokyo, Japan).

### Estimation of Maleimide Density on NP-M

The density of maleimide groups on the surface of NP-M for the conjugation of di-scFv-c was estimated by adding excess amount of cysteine (150 molecules/bead) NP-M in 0.1 M sodium phosphate (pH 7) at room temperature. Free cysteine was then measured by a reaction with 2,4-dinitrothiocyanatebenzene (DNTB), as previously published. (Creighton T E. Protein Structure: A practical Approach. 1989; In Creighton, T. E.:155.) The maleimide functional per particle of NP-M was equivalent to number of cysteine that reacted per particles of NP-M.

Selection of anti-MUC-1 di-scFv-c

The selected di-scFv-c protein was produced in Escherichia coli HB2151 in shaker flasks. (Lu D, X Jimenez, H Zhang, et al. Complete inhibition of vascular endothelial growth factor (VEGF) activities with a bifunctional diabody directed against both VEGF kinase receptor, fms-like tyrosine receptor and kinase insert domain-containing receptor. Cancer Res 2001; 61:7002.) The purified di-scFv-c proteins were analyzed in both 4-12% sodium dodecyl sulfate polyacrylamide gele electrophoresis (SDS-PAGE) gel and 4-12% native gel electrophoresis followed by Coomassie blue staining. Western blot analysis (Takemura S I, R Asano, K Tsumoto, et al. Construction of a diabody (small recombinant bispecific antibody) using a refolding system. Protein Eng 2000; 13:583.) was performed with horseradish peroxidase (HRP)-conjugated anti-E Tag antibody. Further purification was performed by 1×32 cm Sephadex G75 in PBS column chromatography (Pharmacia Biotech, Piscataway, N.J.).

Conjugation of DOTA-Bz-NCS with di-scFv-c and Radiolabeling

DOTA-Bz-NCS was conjugated to di-scFv-c as DOTA-di-scFv-c, with slight modifications, as previously described. (Yazaki P J, A M Wu, S W Tsai, et al. Tumor targeting of radiometal labeled anti-CEA recombinant T84.66 diabody and t84.66 minibody: comparison to radioiodinated fragments. Bioconjug Chem 2001; 12:220.) Briefly, DOTA-Bz-NCS and di-scFv-c were combined in 0.1 M tetramethylammonium phosphate, pH 8.5, at final concentrations of 1 mM, and 0.05 mM, respectively, incubated at 37° C. for 60 minutes, and purified by molecular sieving column chromatography. To prepare radioimmunoconjugate (RIC), ¹¹¹In and DOTA-di-scFv-c was combined in 0.1 M ammonium acetate, pH 5.3, and incubated 30 minutes at 37° C. To scavenge nonspecifically bound ¹¹¹In, ethylenediaminetetraacetic acid (EDTA) was added to a final concentration of 10 mM for 15 minutes at room temperature (RT). The RIC was purified by Sephadex G25 molecular sieving chromatography.

### Preparation of RINP

The NP-M suspension (2 ml, 25 mg/ml, PBS), di-scFv-c (210 ul, 2.5 mg/ml, PBS) and ¹¹¹In-DOTA-di-scFv-c (220 μL, 270 μCi/105 μg, PBS) were mixed (pH 6.8) and kept under constant shaking for 4 h at RT. The RINP reaction mixture was washed three times with PBS buffer on a magnetic separation column and the product eluted with 2 ml of PBS. To this, 0.25 ml of cysteine (2.5 mM in PBS) was added and shaken at RT for 30 minutes to quench remaining active sites. This mixture was washed three times with PBS and eluted with 1 ml of PBS.

### Preparation of Radionanoparticles (RNPs)

The DOTA-benzyl-NH₂ was dissolved in 0.1 mM ammonium acetate (pH 5.3) and mixed with ¹¹¹In-chloride in 0.05 mM HCl (0.2 GBq) buffered to a final pH of 5.3 in 0.1 mM ammonium acetate. Radiolabeled ¹¹¹In-DOTA-Bz-NH₂ was conjugated to COOH functionalized NP (20 nm) via amide linkage. Prior to conjugation, 1-Ethyl-3-(3-dimethylaminopropyl)-carbodiimide (3 mg) and N-hydroxysuccinimide, 6 mg in 0.5 mL of 0.1 mM MES buffer, were mixed with 25 mg/2 mL of spio beads. This suspension was incubated for 1 hour at room temperature with continuous mixing, placed into a 3,400 MW cutoff dialysis bag (3 mL), and dialyzed against 4 L of saline for 1 hour. ¹¹¹In-DOTA-benzyl-NH₂(1.5×10¹⁶ molecules; 5 mCi/20 μg/0.5 mL) was transferred into 0.5 mL of 0.1 mM MES buffer, mixed with the activated NP suspension, and incubated for 2 hour at room temperature with continuous mixing (reaction ratio was 6×10¹⁴ molecules of DOTA/mg of NP). The conjugated ¹¹¹In-DOTA-Bz-NP, 3 mL, was again placed into a dialysis bag (3,400 MW cutoff) and dialyzed against 4 L of saline at room temperature for 1 hour. The dialyzed product was mixed with 1.0 mL of 25 mM glycine and mixed for 15 minutes to quench remaining active sites on the particle surface. The conjugated suspension was applied to the high-gradient magnetic field column separator using saline as both washing buffer and final eluent. The final suspension was collected from the magnetic column after removing it from the magnetic field. The specific activity of the final product was 200 μCi/20 mg/2 mL.

### Characterization of RINP

Reduced 4-12% SDS-PAGE stained by Coomassie blue was used to identify the ¹¹¹In-DOTA-di-scFv-c, di-scFv-c, and RINP before and after purification. CAE demonstrated electrophoresis purity and homogeneity of purified RINP. Concentration of iron per milligram of RINP was measured against reference standard by spectrophotometric method previously published. (Josephson L, C H Tung, A Moore, et al. High-efficiency intracellular magnetic labeling with novel superparamagnetic-Tat peptide conjugates. Bioconjug Chem 1999; 10:186) ¹¹¹In-DOTA-Bz-di-scFv concentrations on RINP were calculated from quantitation of specific activity of the RINP (μCi/mg). The amount of di-scFv-c conjugated to NP was estimated by direct and indirect methods. Estimation of di-scFv-c by direct method was performed by measuring radioactivity of the RINP and by known specific activity of the ¹¹¹In-di-scFv-c and corresponding proportion of the non radioactive di-scFv-c. The indirect estimation of di-scFv-c conjugated to NP was based on measuring of di-scFv-c present in all the washings, after purification of final RINP and subtracting this amount from the total amount of di-scFv-c used for the conjugation of beads by SEC-3000 high performance liquid chromatography (HPLC) at UV-280 nm.

### Cell Culture

HBT3477 expressing MUC-1, human breast cancer cells and COS 7, non-MUC-1 expressing, green monkey kidney cells, and DU-145 moderately MUC-1 expressing prostate cancer cells were obtained from the American Type Culture Collection (Manassas, Va.). Cells were grown and maintained in their recommended media (Gibco, Invitrogen Corporation, Carlsbad, Calif.). Viability was determined by trypan blue exclusion before cell binding assays. MUC-1 positive HBT 3477 and DU145 cells were grown to 90% confluence. HBT 3477 cells were scraped and had 97% viability by trypan blue exclusion. COS7 and DU145 were trypsinized, washed, and had 99% viability by trypan blue exclusion.

### In Vitro Cell Binding Assay of RINP

The ¹¹¹In-DOTA-di-scFv-c, RINP and viable cells (2×10⁷ cells/ml) were diluted to the desired concentrations in PBS (pH 7) to find the maximal binding percentage corresponding to the reported method. (Winthrop M D, S J DeNardo, H Albrecht, et al. Selection and characterization of Anti-MUC-1 scFvs intended for targeted therapy. Clin Cancer Res 2003; 9:3845s.) ¹¹¹In-DOTA-di-scFv-c (10 ng) and RINP each (50 μL, 0.04 mg/mL) were added to triplicate tubes. One million cells, 50 μl were added to the triplicates, vortexed, and incubated for 1 hour at room temperature. The cells were pelleted by centrifugation at 300×g for 10 minutes, and the supernatant and cells were counted. The percentage bound was calculated as follows: (cell bound cpm/[cell cpm+supernatant cpm])×100. The binding effect of ¹¹¹In-DOTA-di-scFv-c and RINP were estimated over the time period by incubation with separation analysis at 1, 2, 4, 12 and 24 hours.

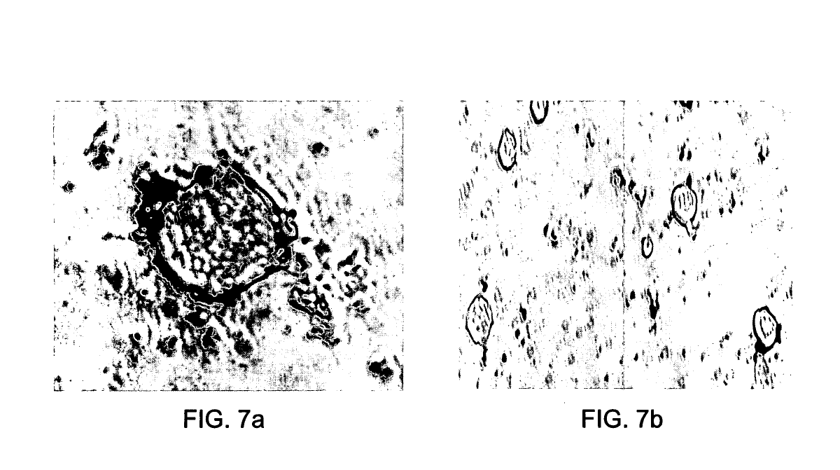

RINP binding is shown in FIGS. 7a and 7b. ¹¹¹In-DOTA-di-scFv-NPs with HBT3477 cells and DU145 cells were mixed and kept at 1 h incubation. Bioprobes were stained with Prussian blue stain and it was analyzed under microscope with 40× magnification. HBT and DU145 cells were binds with anti-Muc-1di-scFv-NPs. Binding effect of RINP was less at DU145 cells compared with HBT (right slide).

### Immunoreactivity of RINP and RNP (Negative Control)

An additional live cell binding assay was carried out with RINP and the RNP (¹¹¹In-DOTA-Bz-NP) control to compare the targeting effect of RINP and just RNP alone. RINP and RNP were tested at 10 and 1 ng equivalent of di-scFv at NP (2 and 0.2 μg of NP) with HBT 3477 (MUC-1 expressed) cells and COS7 (non MUC-1 expressed) cells in a total volume of 150 μl at room temperature for 1 hour. Bound (cell pellets) and free (supernatants) were separated by centrifugation at 300×g for 10 minutes and counted to determine % bound.

### Whole Body Autoradiography (WBAR)

WBAR was conducted, as previously described. (Fand I and W P McNally. The technique of whole-body autoradiography. 1981; 1:1. DeNardo G L, L A Kroger, S J DeNardo, et al. Comparative toxicity studies of yttrium-90 MX-DTPA and 2-IT-BAD conjugated monoclonal antibody (BrE-3). Cancer 1994; 73:1012.) Twenty four hours after tail vein injection of 50 μg/50 μCi/200 μL/5 mg of RINP mice were anesthetized using an intravenous injection of 50 mg/ml of 100 μl aqueous solutions of sodium pentobarbital, then flash frozen in a hexane, dry ice bath. The frozen mice were embedded in 4% carboxymethylcellulose and sagittal sections were generated with a Leica Polycut at −20° C. Sections of 50 μm thicknesses were obtained to show tumors, spleen, kidney, liver and the midline of the vertebral column. Radiographs of the sections were prepared by exposing the sections to x-ray film (Kodak BioMax MS, Rochester, N.Y.).

### Pharmacokinetic Study

Female (8-9 week old), athymic Balb/c nu/nu mice (Harlan Sprague Dawley, Inc., Frederick, Md.) were maintained according to University of California animal care guidelines on a normal diet ad libitum and under pathogen-free conditions. HBT 3477 cells were harvested in log phase; 3.0×10⁶ cells were injected subcutaneously on both sides of the abdomen of mice for the PK studies. All studies were carried out 2 to 4 weeks after tumor implantation. PK studies were performed using RINP doses of 15-20 μCi on 2.2 mg RINP and injected iv into a lateral tail vein with an additional 50 μg di-scFv-c in 200 μl saline.

PK studies were performed using 3 mice (6 tumors) at each time point and sacrificed at 4, 24, 48 hours, respectively. RINP doses were injected iv in 200-250 μl saline. Whole body and blood activity was measured immediately, and again 1 and 4 hours, and 1, and 2 days at the time of sacrifice. Values were expressed as a percent of injected dose (% ID). Blood activity, expressed as % ID/ml, was determined by counting 2 μl blood samples, collected at 5 min, 1, 4, 24 and 48 hours after injection in a gamma well counter (Pharmacia LKB, Piscataway, N.J.). The mice were sacrificed and organs and tissue samples collected. Activity, expressed as % ID/g, was measured in a gamma well counter in a manner similar to that used for blood activity. (DeNardo S J, Burke P A, Leigh B R, et al. Neovascular targeting with cyclic RGD peptide (cRGDf-ACHA) to enhance delivery of radioimmunotherapy. Cancer Biother Radiopharm 2000; 15:71.)

### Results

### Preparation, Characterization, and Quality Assurance of RIC and RINP

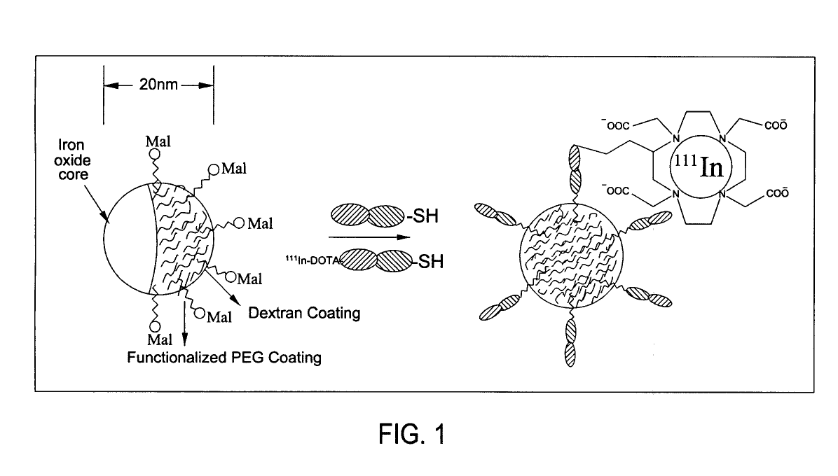

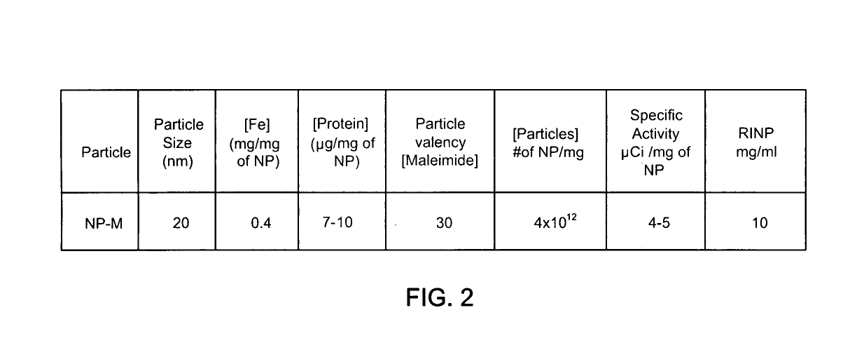

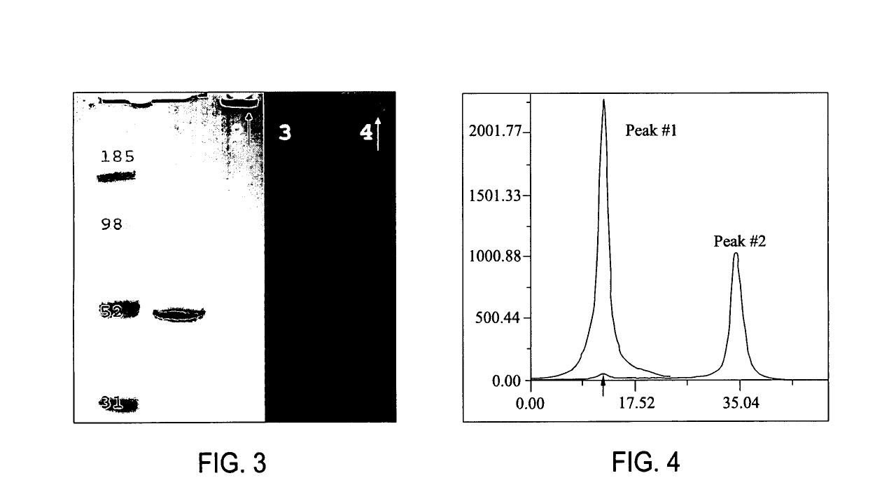

A schematic of an RINP is depicted in FIG. 1. The RINP was prepared by site specific conjugation of anti-MUC-1 di-scFv-c to maleimide functionalized NP. The maleimide functionality per particle of NP-M was 20-30 per NP-M. Characterization of the RINP is given in FIG. 2. The purified RIC was evaluated by CAE, HPLC and PAGE prior to conjugation of NP-M. The RIC was greater than 90% monomeric by CAE and PAGE (See FIG. 3). The specific activity of the RIC was 0.5 μCi/μg. The purified RINP was a dark brown and homogeneous suspension and stayed at the origin of PAGE (See FIG. 3) due to high molecular weight; no unbound di-scFv-c or ¹¹¹In-DOTA-di-scFv was observed. CAE 11 and 45 minutes test showed that the RINP was greater than 90% monomeric with no aggregation or unbound free di-scFv (See FIG. 4). The specific activity of the final purified RINP was 4-5 μCi/7-10 μg of di-scFv/mg of NP. The concentration of di-scFv-c conjugated to NP estimated by subtracting the total proteins in washings after conjugation and purification of RINP from the total protein used to conjugate to NP-M yielded 9-10 μg di-scFv/mg bead. The amount of protein conjugated to RINP yielded 7-10 μg di-scFv/mg of NP calculated by specific radioactivity. Thus estimation of di-scFv conjugated to each NP-M ranged from 20-30 di-scFv molecules per bead.

### Quality Assurance of Radioimmunoconjugate and RINP

The table in FIG. 2 indicates the specification of new RINP. One sixth of the total amount of di-scFv-c used to link SPIO-NP-M was conjugated to DOTA-Bz-NCS and it was radiolabeled with In-111 to trace the RINP. This unmodified di-scFv-c and ¹¹¹In-DOTA-di-scFv-c mixture used in this study was characterized as >90% monomeric by PAGE (see FIG. 3, lane 1). The ¹¹¹In-DOTA-di-scFv-c was purified and assayed on CAE and HPLC, prior to mixing with unmodified di-scFv-c for NP conjugation. The RIC mixture ¹¹¹In-DOTA-di-scFv-c(1 part)+di-scFv-c(5 parts) showed greater than 90% monomeric analyzed by PAGE (see FIG. 3, lanes 1 and 3) and HPLC and its specific activity was 0.42 uCi/ug. FIGS. 3 and 4 of PAGE and CAE analysis of purified RINP was >90% monomeric (FIG. 3, lanes 2 and 4). FIG. 3, lane 2 brown band of RINP was not migrated due to high molecular weight, also no unbound di-scFv-c or ¹¹¹In-DOTA-di-scFv was observed. The specific activity of the final purified RINP was 3-5 μCi/7-8 ug of di-scFv/mg of NP. The di-scFv-c conjugated to NP was estimated by indirect method by subtracting the total proteins in washings after conjugation and purification of RINP from the total protein used to conjugate the NP. Also decay corrected specific activity of the RINP was also used to estimate the amount of protein conjugated to RINP. Thus estimation di-scFv conjugated to each NP was accounted as 25-30 molecules per bead.

FIG. 3 shows ¹¹¹In-DOTA-Bz-di-scFv and ¹¹¹In-DOTA-Bz-di-scFv-NP on 4-12% PAGE spectrum Left: CB stained and Right: same PAGE scanned on Fuji Imager for radioactivity detection. Lanes #1, and 3, ¹¹¹In-DOTA-Bz-di-scFv-c; lanes # 2 and 4, RINP used for animal study. In lanes 2 and 4 RINPs are stayed at origin due to high molecular weight.

FIG. 4 shows quality assurance of the RINP (¹¹¹In-DOTA-Bz-di-scFv-NP) on CAE 11 min (peak #1) and 45 min (peak #2) and showed >90% monomeric.

### In Vitro Cell Biding Assay

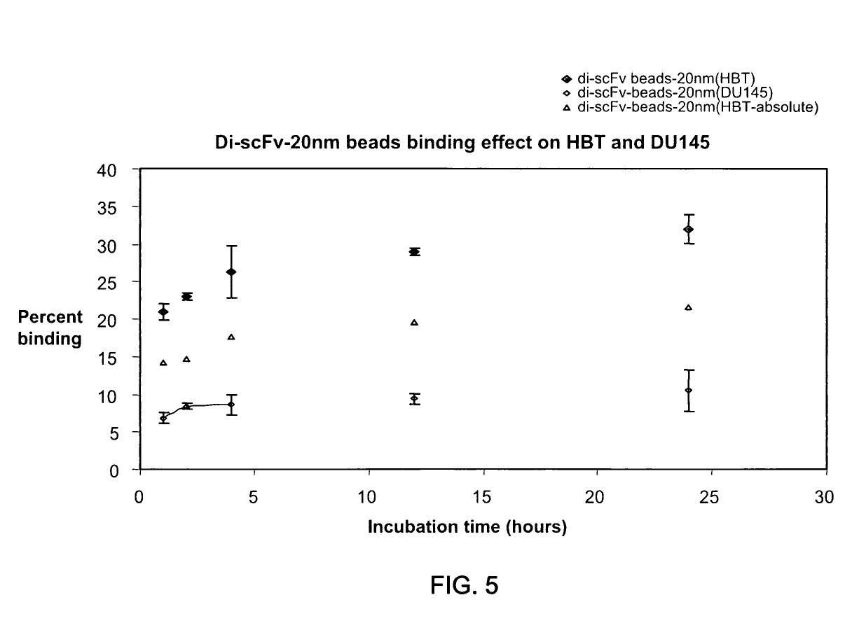

Compare to control ¹¹¹In-DOTA-di-scFv-c the bioprobes showed relative binding of >80% (HBT 3477) and >60% (DU145). The absolute binding effect of RINP 25-30% on HBT 3477 is shown in FIG. 5. The percentage bound were tested at different time intervals of incubation to estimate the binding affinities over the time. RINP had 22-25% specific binding to HBT 3477 and 10-15% to DU145 cells (see FIG. 5). NPs alone bound equal to background (data not shown).

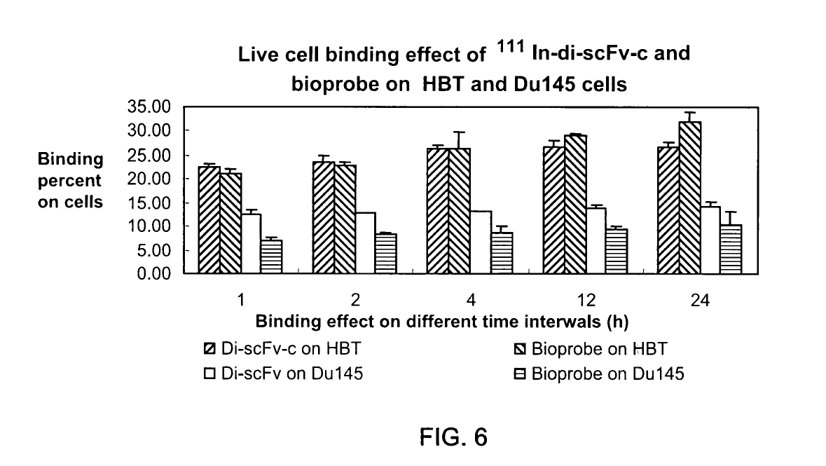

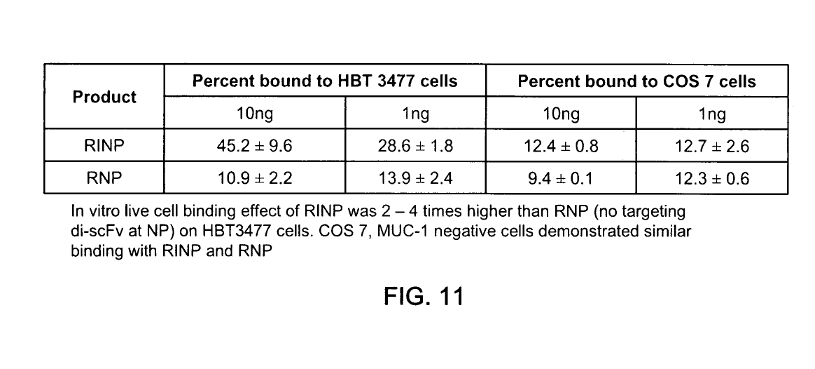

The percent bound of RINP and RIC on HBT3477 and DU145 cells at various time intervals over 24 hours incubation is shown in FIG. 6. Relative binding of RINP, compared to RIC was greater than 80% and 60% with HBT3477 and DU145 cells respectively at 1 h incubation. After 24 hours incubation the RINP had 125% relative binding to HBT3477 cells compared to RIC. The binding of RNP alone showed less than 12%. Both RINP and RNP showed similar binding (FIG. 11) on the control COS 7 cells demonstrating relatively low non specific binding.

### WBAR and PK in Mice

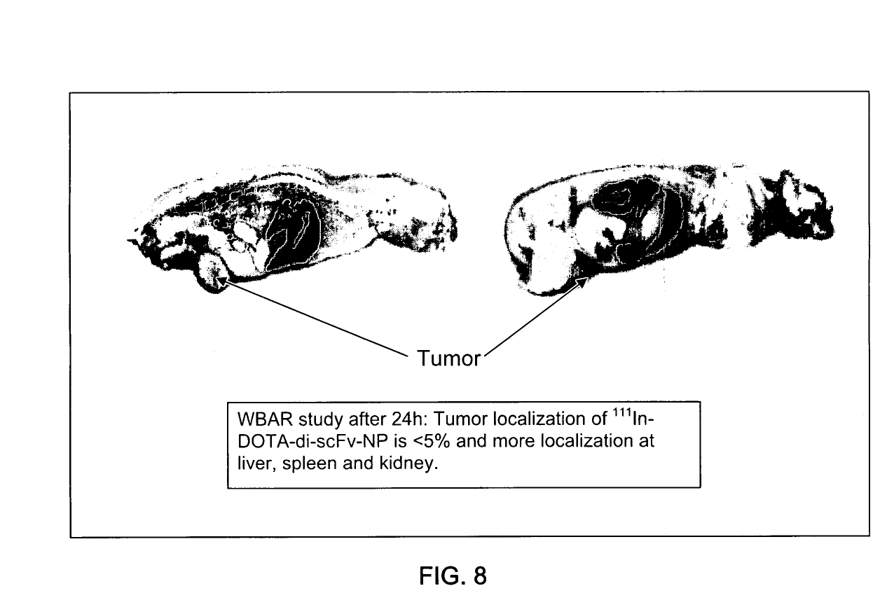

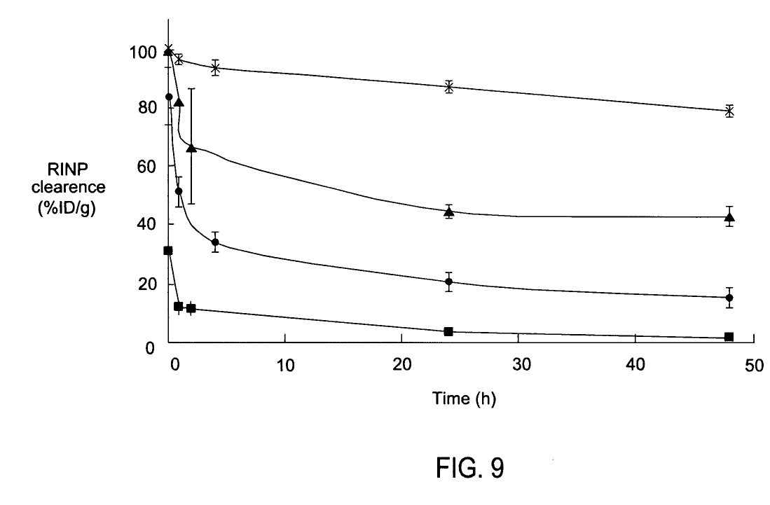

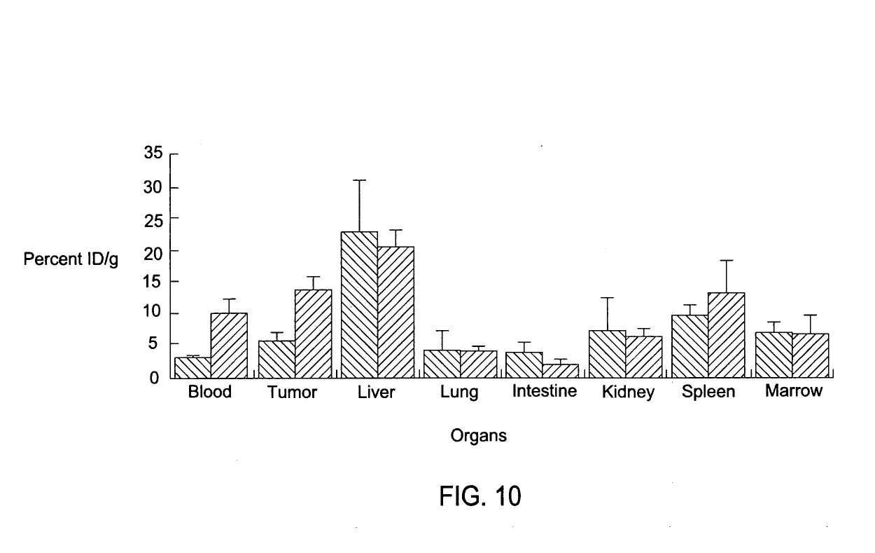

The WBAR was obtained from a mouse sacrificed at 24 hours after injection of RINP (See FIG. 8). Blood and body clearances of the RINP (FIG. 9) and mean RINP concentrations (% ID/g) in lung, kidney and marrow (FIG. 10) were compared to ¹¹¹In-ChL6-NP as previously reported. (DeNardo S J, G L DeNardo, A Natarajan, et al. Thermal dosimetry predictive of efficacy of 111In-ChL6 nanoparticle AMF-Induced thermoablative therapy for human breast cancer in mice. J Nucl Med 2007; 48:437.)

Mean concentrations of RINP at the tumor in the PK study were between 4-5% ID/g studied over 2 days. The mean concentrations of bioprobe (from previous report) and RINP in lung, kidney, spleen and marrow were similar (FIG. 10). However, the concentrations of RINP in liver were slightly higher than the bioprobes. After 24 hours the RINP concentrations in blood was less than 5% compared to 20% with bioprobes; the whole body concentrations of RINP level remained relatively constant after 24 hours (FIG. 10).

Hyperthermia as a therapeutic modality is appealing because it may enhance the effects of many other therapies. However, almost all approaches to deliver hyperthermia have previously resulted in heating not only the tumor, but also healthy tissue. Achieving tumor cell focused hyperthermia is a challenge. The degree of success using this approach depends on the design of targeting NP and number of ligands per NP, size, resulting PK and response to AMF. The present invention may address several issues by applying two specific modifications to the prior ¹¹¹In-ChL6-NP; a) replacing 150 kDa MAbs with 50 kDa di-scFvs to increase number of binding ligands per NP and b) utilizing the cysteine engineered on the di-scFv-c for site-specific conjugation to NP. For example the number of di-scFv-c conjugated to NP was 20-30 molecules as compared to 4-5 MAb molecules per bead. The limit to the number of MAb that conjugated to carboxylate activated NP by may be due to steric hindrance.

Anti-MUC-1 di-scFv-c are well established small antigen-binding proteins that serve as modules in multivalent tumor-targeting constructs designed to enhance radioimaging and therapy. (Moore A, Z Medarova, A Potthast, et al. In vivo targeting of underglycosylated MUC-1 tumor antigen using a multimodal imaging probe. Cancer Res 2004; 64:1821.) Selection and engineering of anti MUC-1 di-scFv-c and PEG maleimide for site-specific thiol conjugation has been reported. (A Natarajan, C Y Xiong, H Albrecht, et al. Characterization of Site-Specific ScFv PEGylation for Tumor-Targeting Pharmaceuticals. Bioconjug Chem 2005; 16:113.) A similar approach used to conjugate radiolabeled di-scFv-c to maleimide functionalized iron oxide NPs demonstrated stable site-specific attachment, excellent homogeneity and good immunoreactivity. Carbodiimide chemistry may be used to form an amide bond between NH₂ of MAb and COOH groups of nanoparticles (DeNardo S J, GL DeNardo, L A Miers, et al. Development of tumor targeting bioprobes ((111)In-chimeric L6 monoclonal antibody nanoparticles) for alternating magnetic field cancer therapy. Clin Cancer Res 2005; 11:7087s.) resulting in amine groups at the MAb forming covalent linkage without any control over the conjugation strategy. The advantage of the site-specific conjugation of di-scFv-c to NP showed controlled coupling, defined molecular conformation and increased binding efficiency over di-scFv alone. The advantage of antibody fragments over intact MAb is to minimize immunogenicity and to circumvent clearance via Fc receptor-mediated mechanisms. (Kirpotin D B, D C Drummond, Y Shao, et al. Antibody targeting of long-circulating lipidic nanoparticles does not increase tumor localization but does increase internalization in animal models. Cancer Res 2006; 66:6732.)

The new RINP using anti-MUC-1 antibody fragments was intentionally designed to target a wide range of cancers for AMF therapy, since MUC-1 is over expressed in most human epithelial cell adenocarcinomas, including greater than 90% of human breast cancer (Avichezer D, J Taylor-Papadimitriou, R Arnon. A short synthetic peptide (DTRPAP) induces anti-mucin (MUC-1) antibody, which is reactive with human ovarian and breast cancer cells. Cancer Biochem Biophys 1998; 16:113. Hayes D F, R Mesa-Tejada, L D Papsidero, et al. Prediction of prognosis in primary breast cancer by detection of a high molecular weight mucin-like antigen using monoclonal antibodies DF3, F36/22, and CU18: a Cancer and Leukemia Group B study. J Clin Oncol 1991; 9:1113. Nacht M, A T Ferguson, W Zhang, et al. Combining serial analysis of gene expression and array technologies to identify genes differentially expressed in breast cancer. Cancer Res 1999; 59:5464.) and greater than 50% of all cancers in humans. (Greenlee R T, T Murray, S Bolden, et al. Cancer statistics, 2000. CA A Cancer Journal for Clinicians 2000; 50:7.)

The newly developed RINP was able to bind anti-MUC-1 expressing cancer cells, and the relative immunoreactivity was greater than 80% compared to ¹¹¹In-DOTA-di-scFv-c at 2 hours. The in vitro binding efficiency of the RINP was time-dependent and comparable to RIC. The RINP preparation had excellent binding properties in terms of anti-MUC-1 expressing tumor cells compared to non MUC-1 tumor cells.

The PK and WBAR study confirms the targeting of RINP to breast cancer tumors in vivo. The calculated iron content uptake from PK and WBAR (% ID) was 5 μg/g of tumor equivalent to 5×10¹⁰ particles. The concentration of the iron delivered at tumor site and the predicted heat energy from externally applied AMF, would be sufficient to kill or inactivate many cells. This was demonstrated previously by electron microscopy which provided strong evidence that cells were killed by necrosis. (DeNardo S J, G L DeNardo, L A Miers, et al. Development of tumor targeting bioprobes ((111)In-chimeric L6 monoclonal antibody nanoparticles) for alternating magnetic field cancer therapy. Clin Cancer Res 2005; 11:7087s.) Compared to a previously published study using ¹¹¹In-ChL6-NP, the present RINP showed better blood and body clearance. However, tumor uptake of RINP was 50% less than ¹¹¹In-ChL6-NP. This may be due to faster clearance of RINP from blood (90%) and whole body (32%) at 2 hours, compared to bioprobe clearance from blood (40%) and whole body (5%) at 2 hours. The RINP concentrations of whole body was 68% compared to bioprobe 95% after 2 h; thus the RINP available over time in the blood at tumor site, was drastically reduced which could be the reason for the reduced tumor uptake by RINP compared to bioprobes. The RINP uptake by various organs such as kidney, lung and marrow was comparable, lower in spleen, and slightly higher in liver compared to ¹¹¹In-ChL6-NP (FIG. 9). Thus the new RINP linked to antibody fragments has two key features: a) capability to target the anti-MUC-1 expressing cancer tissues and b) the radioactive tag allowing quantification of the particle uptake in tumor in vivo. These features may enable effective combinations with other therapeutic modalities.

In conclusion, a novel RINP has been developed using site-specific coupling methods for the preparation of radiolabeled di-scFv linked NP to target cancer cells for the focused hyperthermia by AMF. In this preparation, NPs were attached to di-scFv-c at a well-defined site, and the number of antibody fragments per bead was 20-30 molecules with 7-10 μg/mg of RINP. The binding effect of RINP is comparable to unmodified ¹¹¹In-di-scFv-c. RINPs binding of cancer cells were time-dependent and showed increased binding over time compared to ¹¹¹In-di-scFv-c alone. This appears to be the first report on the site-specific conjugation of radiolabeled di-scFv-c and maleimide-functionalized nanoparticles for AMF therapy. PK and WBAR study showed the RINP up take in breast cancer tumor was confirmed in vivo.

### Example

### Purpose

Development of di-scFv conjugated bioprobes (¹¹¹In-DOTA-Bz-di-scFv-20 nm-particles) for animal study.

### Background

Conjugation of ¹¹¹In-DOTA-Bz-di-sc-Fv to Nanomag®-D-Spio particles using thiol-maleimide chemistry to target anti MUC-1 antigens by In vitro and In vivo study.

### Materials and Methods

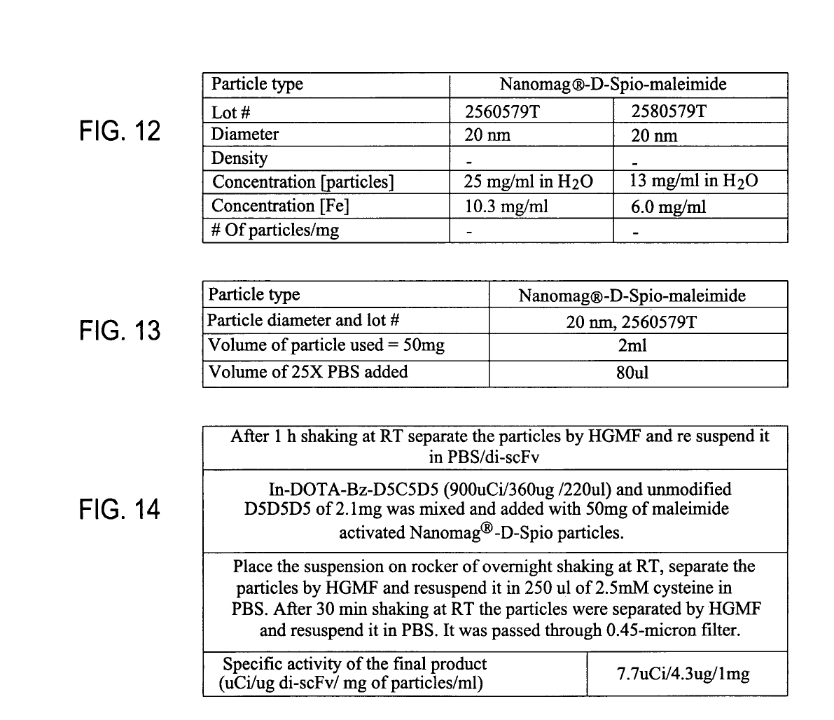

Nanomag®-D-Spio particles (maleimide function) suspension 20 nm 25 mg/ml (lot# 2560579T) was buffer exchanged with PBS (FIG. 12).

25 mM cysteine in PBS, ¹¹¹In-DOTA-Bz-D5C5D5-c specific activity of 25 uCi/ug and HGMF (High grade magnetic field separator)

The final suspension of ¹¹¹In-DOTA-Bz-di-scFv-Nanomag®-D-Spioparticles (20 nm) collected from the HGM field was homogenous dark brown liquid (see FIG. 14). The specific activity of this lot was given in FIG. 13. To check the purity of the product 2 ul of the sample was allowed to elute on CAE at 11 and 45 minutes. For protein characterization 15 ul of sample was applied on PAGE.

### Results

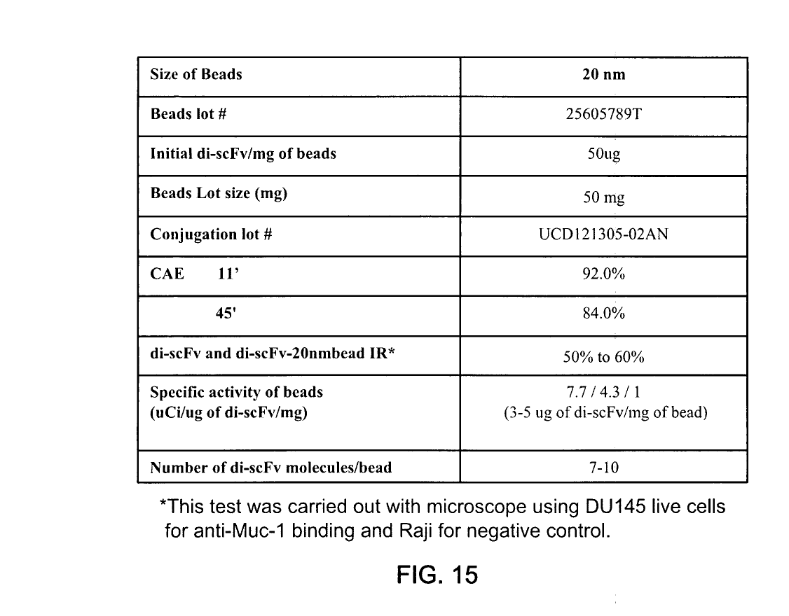

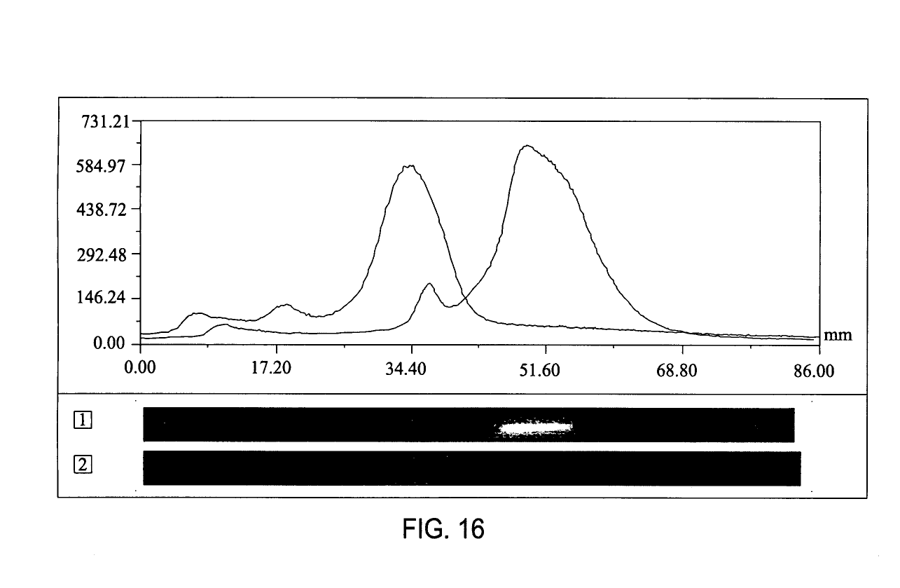

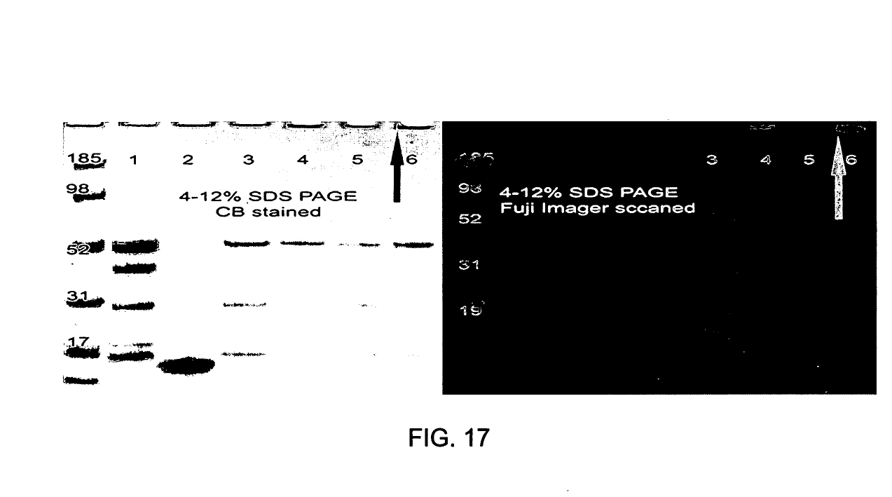

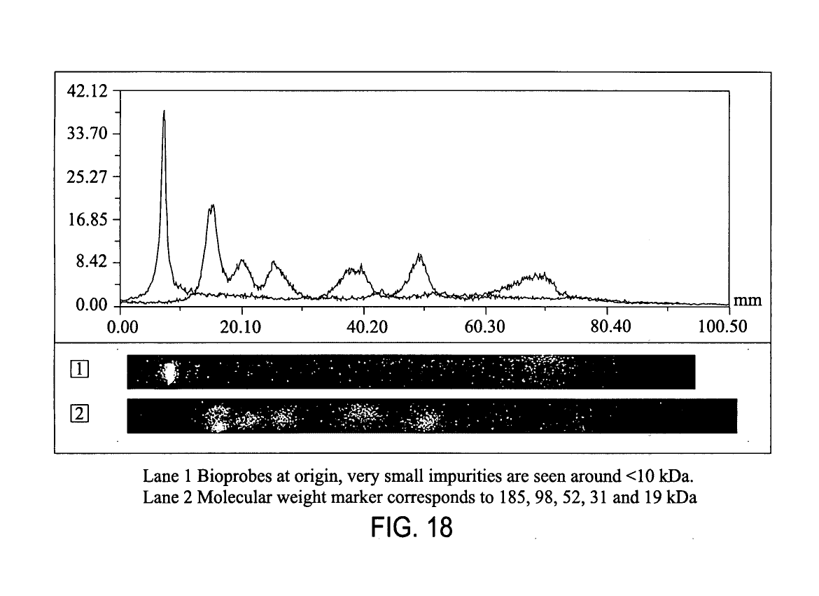

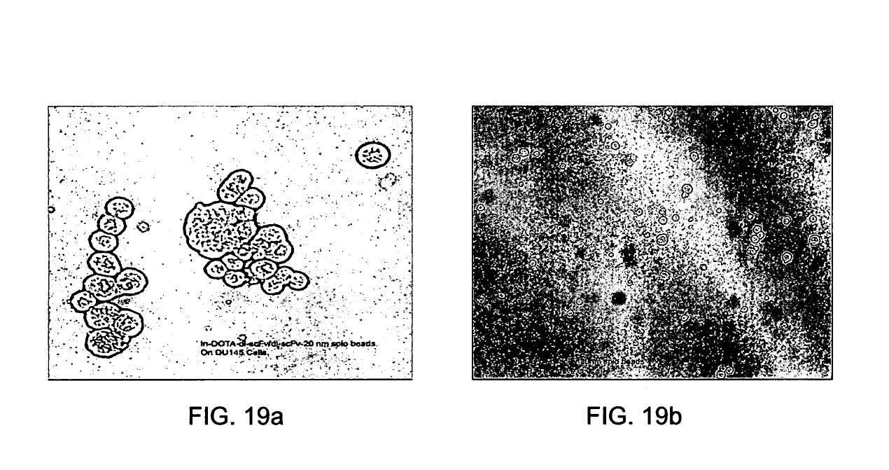

QA of the ¹¹¹In-DOTA-Bz-di-scFv-Nanomag®-D-Spioparticles (20 nm) tested by CAE were shown in FIGS. 15 and 16. The binding effect of DU-145 cells on beads was shown in FIG. 19a. The Raji cells, shown in FIG. 18b, did not bind. FIGS. 17 and 18 show 4-12% SDS-PAGE spectrum of beads stained with coomassie blue for protein identification and also scanned by Fuji Imager to identify radioactivity of the beads.

### Discussion

The anti-MUC-1 ¹¹¹In-DOTA-Bz-di-scFv/di-scFv was conjugated with maleimide activated Nanomag®-D-Spio particles (20 nm) with established protocol. ¹¹¹In-DOTA-Bz-di-scFv/di-scFv-Nanomag®-D-Spio particles (20 nm) were purified by magnetic column to wash off unbound ¹¹¹In and ¹¹¹In-DOTA-Bz-di-scFv/di-scFv. The CAE of the final product at 11 and 45 minutes was >75% monomeric. Conjugation of ¹¹¹In-DOTA-Bz-di-scFv with PEG-maleimide group of Nanomag®-D-Spio particles was 7-10 di-scFv per particle. Protein concentrations were estimated against its radioactivity.

### CONCLUSION

¹¹¹In-DOTA-Bz-di-scFv/di-scFv-20 nm Nanomag®-D-SPIO particles have 7-10 di-scFv molecules/bead (i.e., 3-5 ug di-scFv/mg of beads).

It should be understood, of course, that the foregoing relates to exemplary embodiments of the invention and that modifications may be made without departing from the spirit and scope of the invention as set forth in the following claims. For example, useful nanoparticles may include nanoparticles having different dimensions and core compositions than the nanoparticles described above just so it is thiol reacting groups activated (e.g. Maleimide, Bromine or other halogens). Additionally, other recombinant proteins or peptides may be used in lieu of scFv or di-scFv as long as the conjugation is through an SH (sulfhydryl group) such as the reactive SH on cysteine described above, but other reactive SH groups may be utilized.

Each of the references cited in the specification above are herein incorporated by reference in their entirety.

We claim:

1. A conjugate comprising:
a nanoparticle; and
at least one ligand having a free thiol group, said free thiol group linked to said nanoparticle.

2. The conjugate of claim 1, wherein the ligand is selected from the group consisting of proteins, polypeptides, antibodies and single chain variable fragments.

3. The conjugate of claim 1, wherein the at least one ligand comprises from 1 to about 50 ligands linked to the nanoparticle.

4. The conjugate of claim 1, wherein the ligand has at least one binding region.

5. The conjugate of claim 4, wherein the thiol group does not interfere with the binding region.

6. The conjugate of claim 1, wherein the thiol group is designed into the ligand to provide site specific linking of the ligand to the nanoparticle.

7. The conjugate of claim 1, wherein the nanoparticle is selected from the group consisting of an iron oxide particle, gold particle and a liposome.

8. A conjugate comprising:
a ligand having multiple specificity; and
a nanoparticle attached to a free cysteine of said ligand.

9. The conjugate of claim 8, wherein the ligand is a recombinant antibody fragment.

10. The conjugate of claim 8, wherein the tumor cell is MUC-1.

11. The conjugate of claim 8, wherein the nanoparticle is a maleimide activated nanoparticle.

12. The conjugate of claim 8, wherein the multiple specificity is dual specificity toward a tumor cell and toward a metal chelating group.

13. A conjugate comprising:
a nanoparticle; and
at least five ligands conjugated to said nanoparticle through a thiol group of the ligand, each of the ligands comprising a bivalent molecule.

14. The conjugate of claim 13, wherein the ligands are antibody fragments.

15. A radio-immuno nanoparticle (RINP) comprising:
a nanoparticle; and
a plurality of radiolabeled ligands attached to said nanoparticle through a thiol group of each of the ligands. such that said RINP has at least ten MUC-1 binding units.

16. The RINP of claim 15, wherein the ligands are antibody fragments.

17. The RINP of claim 15, wherein the nanoparticles comprise magnetic iron oxide particles.

18. The RINP of claim 16, wherein the RINP has at least ten MUC-1 binding units.
